SEQUENCE LISTING (29 sequences)

SEQ ID NO: 1 - DNA, 498 bp, Homo sapiens
  CDS (1)..(498): Human IFN alpha DNA sequence
tgtgatctgcctcagacccacagcctgggtaataggagggccttgatactcctggcacaaatgggaagaatctctcctttctcctgcctgaaggacagacatgactttggattcccccaggaggagtttgatggcaaccagttccagaaggctcaagccatccctgtcctccatgagatgatccagcagaccttcaatctcttcagcacaaaggactcatctgctacttgggaacagagcctcctagaaaaattttccactgaacttaaccagcagctgaatgacctggaagcctgcgtgatacaggaggttggggtggaagagactcccctgatgaatgtggactccatcctggctgtgaagaaatacttccaaagaatcactctttatctgacagagaagaaatacagcccttgtgcctgggaggttgtcagagcagaaatcatgagatccttctctttatcaaaaatttttcaagaaagattaaggaagaaggat

SEQ ID NO: 2 - protein, 166 aa, Homo sapiens
CDLPQTHSLGNRRALILLAQMGRISPFSCLKDRHDFGFPQEEFDGNQFQKAQAIPVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRKKD

SEQ ID NO: 3 - DNA, 696 bp, Homo sapiens
  CDS (1)..(696): Human Fc DNA sequence
gagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 4 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 27 bp, Homo sapiens
  Forward PCR primer
cccgggtaaatgtgatctgcctcagac

SEQ ID NO: 6 - DNA, 26 bp, Homo sapiens
ctcgagtcaatccttcctccttaatc

SEQ ID NO: 7 - protein, 162 aa, Homo sapiens
  IFN alpha consensus sequence wherein, Xaa at any position besides positions 24,31,70 and 129 represents any amino acid.
CDLXXXXXLXXXXXLXXXXXMXXXSPXXCLXXRXDFXXPXEXXXXXQXXXXQAXXVLXXXXQQXXXLFXXXXXSAXWXXTLLXXXXXXLXQQLXDLXXCXXXXXXXXXXXLXXVXXXLXVXXYFXXIXXYLXXKXXSXCAWEXXXXXXMRXXSXXXXLXXRL

SEQ ID NO: 8 - protein, 166 aa, Homo sapiens
  Human IFN alpha-1 protein
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 9 - protein, 165 aa, Homo sapiens
  Human IFN alpha-2 protein
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 10 - protein, 166 aa, Homo sapiens
  Human IFN alpha-4 protein
CDLPQTHSLGNRRALILLAQMGRISHFSCLKDRHDFGFPEEEFDGHQFQKTQAISVLHEMIQQTFNLFSTEDSSAAWEQSLLEKFSTELYQQLNDLEACVIQEVGVEETPLMNVDSILAVRKYFQRITLYLTEKKYSPCAWEVVRAEIMRSLSFSTNLQKRLRRKD

SEQ ID NO: 11 - protein, 166 aa, Homo sapiens
  Human IFN alpha-5 protein
CDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 12 - protein, 166 aa, Homo sapiens
  HUman IFN alpha-6 protein
CDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 13 - protein, 166 aa, Homo sapiens
  Human IFN alpha-7 protein
CDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 14 - protein, 166 aa, Homo sapiens
  Human IFN alpha-8 protein
CDLPQTHSLGNRRALILLAQMRRISPFSCLKDRHDFEFPQEEFDDKQFQKAQAISVLHEMIQQTFNLFSTKDSSAALDETLLDEFYIELDQQLNDLEVLCDQEVGVIESPLMYEDSILAVRKYFQRITLYLTEKKYSSCAWEVVRAEIMRSFSLSINLQKRLKSKE

SEQ ID NO: 15 - protein, 166 aa, Homo sapiens
  Human IFN alpha-10 protein
CDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 16 - protein, 170 aa, Homo sapiens
  Human IFN alpha-14 protein
CSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSFSFSTNLQKRLRRKD

SEQ ID NO: 17 - protein, 166 aa, Homo sapiens
  Human IFN alpha-16 protein
CDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 18 - protein, 166 aa, Homo sapiens
  Human IFN alpha-17 protein
CDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 19 - protein, 166 aa, Homo sapiens
  Human IFN alpha-21 protein
CDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 20 - protein, 172 aa, Homo sapiens
  Human IFN delta-1 protein
CDLSQNHVLVGRKNLRLLDEMRRLSPHFCLQDRKDFALPQEMVEGGQLQEAQAISVLHEMLQQSFNLFHTEHSSAAWDTTLLEPCRTGLHQQLDNLDACLGQVMGEEDSALGRTGPTLALKRYFQGIHVYLKEKGYSDCAWETVRLEIMRSFSSLISLQERLRMMDGDLSSP

SEQ ID NO: 21 - protein, 172 aa, Homo sapiens
  Human IFN omega-1 protein
CDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 22 - protein, 166 aa, Mus musculus
  Mouse IFN alpha-1 protein
CDLPQTHNLRNKRALTLLVQMRRLSPLSCLKDRKDFGFPQEKVDAQQIKKAQAIPVLSELTQQILNIFTSKDSSAAWNATLLDSFCNDLHQQLNDLQGCLMQQVGVQEFPLTQEDALLAVRKYFHRITVYLREKKHSPCAWEVVRAEVWRALSSSANVLGRLREEK

SEQ ID NO: 23 - protein, 167 aa, Mus musculus
  Mouse IFN alpha-2 protein
CDLPHTYNLRNKRALKVLAQMRRLPFLSCLKDRQDFGFPLEKVDNQQIQKAQAIPVLRDLTQQTLNLFTSKASSAAWNATLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 24 - protein, 162 aa, Mus musculus
  Mouse IFN alpha-4 protein
CDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKE

SEQ ID NO: 25 - protein, 166 aa, Mus musculus
  Mouse IFN alpha-5 protein
CDLLQTHNLRNKRALTLLVKMRRLSPLSCLKDRKDFGFPQEKVGAQQIQEAQAIPVLSELTQQVLNIFTSKDSSAAWNATLLDSFCNEVHQQLNDLKACVMQQVGVQESPLTQEDSLLAVRKYFHRITVYLREKKHSPCAWEVVRAEVWRALSSSVNLLARLSKEE

SEQ ID NO: 26 - protein, 166 aa, Mus musculus
  Mouse IFN alpha-6 protein
CDLPQTHNLRNKRALTLLVKMRRLSPLSCLKDRKDFGFPQEKVGAQQIQEAQAIPVLTELTQQILTLFTSKDSSAAWNATLLDSFCNDLHQLLNDLQGCLMQQVEIQALPLTQEDSLLAVRTYFHRITVFLREKKHSPCAWEVVRAEVWRALSSSAKLLARLNEDE

SEQ ID NO: 27 - protein, 167 aa, Mus musculus
  Mouse IFN alpha-7 protein
CDLPQTHNLRNKRALTLLVKMRRLSPLSCLKDRKDFGFPQAKVDAQQIQEAQAIPVLSELTQQILNIFTSKDSSAAWNATLLDSVCNDLHQQLNDLQGCLMQEVGVQELSLTQEDSLLAVRKYFHRITVFLREKKHSPCAWEVVRAEIWRALSSSANLLARLSEKKE

SEQ ID NO: 28 - protein, 166 aa, Mus musculus
  Mouse IFN alpha-8 protein
CDLPQTHNLRNKRALTLLVKMRRLSPLSCLKDRKDFGFPQEKVGAQQIQEAQAIPVLTELTQQILALFTSKDSSAAWNATLLDSFCNDLHQLLNDLQGCLMQQVEIQALPLTQEDSLLAVRTYFHRITVFLREKKHSPCAWEVVRAEVWRALSSSAKLLARLNEDE

SEQ ID NO: 29 - protein, 167 aa, Mus musculus
  Mouse IFN alpha-9 protein
CDLPQTHNLRNKKILTLLAQMRRLSPLSCLKDRKDFGFPQEKVDAQQIQEAQAIPVLSELTQQILTLFTSKDSSAAWNATLLDSFCTGLHQLLNDLQGCLMQLVGMKELPLTQEDSQLAMKKYFHRITVYLREKKHSPCAWEVVRAEVWRALSSSVNLLARLSEEKE

(19) United States
(12) Patent Application Publication
(10) Pub. No.: US 2005/0042729 A1
(43) Pub. Date: Feb. 24, 2005

(54) Expression and export of interferon-alpha proteins as Fc fusion proteins

(75) Inventors: Kin-Ming Lo, Lexington, MA (US); Yaping Sun, Norwood, MA (US); Stephen D. Gillies, Carlisle, MA (US)

Correspondence Address: TESTA, HURWITZ & THIBEAULT, LLP, HIGH STREET TOWER, 125 HIGH STREET, BOSTON, MA, 02110, US

(73) Assignee: EMD Lexigen Research Center Corp., Billerica, MA (US)

(21) Appl. No.: 10/953,259

(22) Filed: Sep. 29, 2004

Related U.S. Application Data
(63) Continuation of application No. 09/977,034, filed on Oct. 11, 2001.
Application No. 09/977,034 is a division of application No. 09/575,503, filed on May 19, 2000, now abandoned.
(60) Provisional application No. 60/134,895, filed on May 19, 1999.

(51) Int. Cl. C07H 21/04; C07K 16/18

(52) U.S. Cl. 435/69.1; 435/320.1; 435/328; 530/387.3; 536/23.53

(57) ABSTRACT

Disclosed are nucleic acid sequences, for example, DNA or RNA sequences, which encode an immunoglobulin Fc-Interferon-alpha fusion protein. The nucleic acid sequences can be inserted into a suitable expression vector and expressed in mammalian cells. Also disclosed is a family of immunoglobulin Fc-Interferon-alpha fusion proteins that can be produced by expression of such nucleic acid sequences. Also disclosed are methods of using such nucleic acid sequences and/or fusion proteins for treating conditions, for example, hepatitis, which are alleviated by the administration of interferon-alpha.

### RELATED APPLICATIONS

This application claims priority to U.S. Provisional Application Ser. No. 60/134,895, filed May 19, 1999, the disclosure of which is incorporated herein by reference.

### FIELD OF THE INVENTION

The invention disclosed herein relates to fusion protein expression systems that enhance the production of members of the interferon-alpha class of proteins. More specifically, the invention relates to high level expression and secretion in mammalian cells of Fc fusion proteins, such as immunoglobulin Fc-Interferon-alpha, and the various structural forms and uses thereof.

### BACKGROUND OF THE INVENTION

The interferon-alpha (IFN-alpha) family of proteins has proven to be useful in treatment of a variety of diseases. For example, interferons alpha 2a and 2b (trade names Roferon and Intron A, respectively) have been used in the treatment of chronic hepatitis B, C and D (life-threatening viral diseases of the liver), condylomata acuminata (genital warts), AIDS-related Kaposi's sarcoma, hairy cell leukemia, malignant melanoma, basal cell carcinoma, multiple myeloma, renal cell carcinoma, herpes I and II, varicella/herpes zoster, and mycosis fungoides. The efficacy of treatment regimes containing interferon-alpha prostate cancer and chronic myelogenous leukemia have also been studied.

The human interferon-alpha family is the largest and most complex family of interferons. Members of the interferon-alpha family have similar amino acid sequences that define them as a group distinct from other interferons; i.e., these proteins typically have at least 35% amino acid identity in a typical protein sequence alignment. The SwissProt database contains numerous human interferon-alpha proteins, including the alternatively named interferon-delta and interferon-omega proteins. These proteins typically are synthesized with a leader sequence of about 23 amino acids, and the mature proteins typically have a molecular weight of about 19 kD. Because these proteins are so similar, when interferon-alpha is obtained from a human or other mammalian source and extensively purified, a mixture of isospecies with varying biological activities often are obtained [Georgiadis et al., U.S. Pat. No. 4,732,683]. Similarly, the cDNAs encoding these proteins have sufficiently similar sizes and properties that a single set of procedures can be used to manipulate them for purposes of plasmid construction. Accordingly, it would be useful to have a method for efficiently producing and purifying a single species of interferon-alpha from a mammalian source.

Because of its relatively small size of about 19 kD (Lawn et al. (1981) PROC. NATL. ACAD. SCI. U.S.A. 78: 5435), interferon-alpha can be filtered by the kidney. However, when filtered, interferon-alpha typically is absorbed and metabolized by kidney tubular cells and, therefore, usually is not excreted. According to current clinical practice, formulated interferon-alpha is administered by intramuscular injection, after which its levels in serum decline with a half-life of about 5 hours for interferon-alpha 2a and 2-3 hours for interferon-alpha 2b (PHYSICIANS DESK REFERENCE, 50th edition, 1996: 2145-2147 and 2364-2373).

Furthermore, because of their small size, multiple, frequent injections of interferon-alpha are required (usually daily or 3 times/week), and there can be significant variation in the level of interferon-alpha in the patient. In addition, the injected doses are large, ranging from about 50 micrograms per dose for hairy cell leukemia to 300 micrograms per dose for AIDS-related Kaposi's sarcoma. High levels of circulating interferon-alpha can result in significant side effects, including skin, neurologic, immune and endocrine toxicities. It is thought that the small size of interferon-alpha allows it to pass through the blood-brain barrier and enter the central nervous system, accounting for some of the neurologic side effects. Accordingly, it would be useful to increase the potency and effective serum half-life in patients being treated with interferon-alpha while at the same time minimizing side effects.

Given the high dosage, low efficacy, short serum half-life, difficulties in purification, and side effects of interferon-alpha, there is a need in the art for methods of enhancing the production and improving the pharmacological properties of this therapeutic agent.

### SUMMARY OF THE INVENTION

The present invention features methods and compositions useful for making and using fusion proteins containing interferon-alpha. In particular, the invention features nucleic acids, for example, DNA or RNA sequences, encoding an immunoglobulin Fc-interferon-alpha fusion protein, and methods for expressing the nucleic acid to produce such fusion proteins. The fusion proteins can facilitate high level expression of biologically active interferon-alpha. The fusion protein can be combined with a pharmaceutically acceptable carrier prior to administration to a mammal, for example, a human. Under certain circumstances, the interferon-alpha can be cleaved from the fusion protein prior to formulation and/or administration. Alternatively, nucleic acid sequences encoding the interferon-alpha containing fusion protein can be combined with a pharmaceutically acceptable carrier and administered to the mammal.

It is an object of the invention to provide novel nucleic acid sequences, for example, DNAs and RNAs, which facilitate the production and secretion of interferon-alpha. In particular, the invention provides (i) nucleic acid sequences which facilitate efficient production and secretion of interferon-alpha; (ii) nucleic acid constructs for the rapid and efficient production and secretion of interferon-alpha in a variety of mammalian host cells; and (iii) methods for the production, secretion and collection of recombinant interferon-alpha or genetically engineered variants thereof, including non-native, biosynthetic, or otherwise artificial interferon-alpha proteins such as proteins which have been created by rational design.

Other objects of the invention are to provide polynucleotide sequences which, when fused to a polynucleotide encoding interferon-alpha, encode an interferon-alpha containing fusion polypeptide which can be purified using common reagents and techniques. Yet another object is to interpose a proteolytic cleavage site between a secretion cassette and the encoded interferon-alpha protein such that the secretion cassette can be cleaved from the interferon-alpha domain so that interferon-alpha may be purified independently.

Accordingly, in one aspect, the present invention provides nucleic acid molecules, for example, DNA or RNA molecules, which encode an immunoglobulin Fc region-interferon-alpha fusion protein. The nucleic acid molecule encodes serially in a 5′ to 3′ direction, a signal sequence, an immunoglobulin Fc region, and at least one target protein, wherein the target protein comprises interferon-alpha.

In a preferred embodiment, the immunoglobulin Fc region comprises an immunoglobulin hinge region and preferably comprises at least one immunoglobulin constant heavy region domain, for example, an immunoglobulin constant heavy 2 (CH2) domain, an immunoglobulin constant heavy 3 (CH3) domain, and depending upon the type of immunoglobulin used to generate the Fc region, optionally an immunoglobulin constant heavy chain 4 (CH4) domain. In a more preferred embodiment, the immunoglobulin Fc region lacks at least an immunoglobulin constant heavy 1 (CH1) domain. Although the immunoglobulin Fc regions may be based on any immunoglobulin class, for example, IgA, IgD, IgE, IgG, and IgM, immunoglobulin Fc regions based on IgG are preferred.

The nucleic acid of the invention can be incorporated in operative association into a replicable expression vector which can then be introduced into a mammalian host cell competent to produce the interferon-alpha-based fusion protein. The resultant interferon-alpha-based fusion protein is produced efficiently and secreted from the mammalian host cell. The secreted interferon-alpha-based fusion protein may be collected from the culture media without lysing the mammalian host cell. The protein product can be assayed for activity and/or purified using common reagents as desired, and/or cleaved from the fusion partner, all using conventional techniques.

In another aspect, the invention provides fusion proteins containing interferon-alpha. The fusion proteins of the present invention demonstrate improved biological properties over native interferon-alpha such as increased solubility, prolonged serum half-life and increased binding to its receptor. These properties may improve significantly the clinical efficacy of interferon-alpha. In a preferred embodiment, the fusion protein comprises, in an N- to C-terminal direction, an immunoglobulin Fc region and interferon-alpha, with other moieties, for example, a proteolytic cleavage site, optionally interposed between the immunoglobulin Fc region and the interferon-alpha. The resulting fusion protein preferably is synthesized in a cell that glycosylates the Fc region at normal glycosylation sites, i.e., which usually exist in template antibodies.

In another embodiment, the fusion protein may comprise a second target protein, for example, mature, full length interferon-alpha or a bioactive fragment thereof. In this type of construct the first and second target proteins can be the same or different proteins. The first and second target proteins may be linked together, either directly or by means of a polypeptide linker. Alternatively, both target proteins may be linked either directly or via a polypeptide linker, to the immunoglobulin Fc region. In the latter case, the first target protein can be connected to an N-terminal end of the immunoglobulin Fc region and the second target protein can be connected to a C-terminal end of the immunoglobulin Fc region.

In another embodiment, two fusion proteins may associate, either covalently, for example, by a disulfide bond, a polypeptide bond or a crosslinking agent, or non-covalently, to produce a dimeric protein. In a preferred embodiment, the two fusion proteins are associated covalently by means of at least one and more preferably two interchain disulfide bonds via cysteine residues, preferably located within immunoglobulin hinge regions disposed within the immunoglobulin Fc regions of each chain.

Other objects of the invention are to provide multivalent and multimeric forms of interferon-alpha fusion proteins and combinations thereof.

In another aspect, the invention provides methods of producing a fusion protein comprising an immunoglobulin Fc region and the target protein. The method comprises the steps of (a) providing a mammalian cell containing a DNA molecule encoding such a fusion protein, either with or without a signal sequence, and (b) culturing the mammalian cell to produce the fusion protein. The resulting fusion protein can then be harvested, refolded, if necessary, and purified using conventional purification techniques well known and used in the art. Assuming that the fusion protein comprises a proteolytic cleavage site disposed between the immunoglobulin Fc region and the target protein, the target can be cleaved from the fusion protein using conventional proteolytic enzymes and if necessary, purified prior to use.

In yet another aspect, the invention provides methods for treating conditions alleviated by interferon-alpha or active variants thereof by administering to a mammal an effective amount of interferon-alpha produced by a method of the invention and/or a fusion construct of the invention. The invention also provides methods for treating conditions alleviated by interferon-alpha or active variants thereof by administering a nucleic acid of the invention, for example, a “naked DNA,” or a vector containing a DNA or RNA of the invention, to a mammal having the condition.

In a preferred embodiment, the constructs of the invention can be used in the treatment of a liver disorder, wherein the interferon-alpha by virtue of the immunoglobulin Fc region becomes localized within the liver. The constructs of the invention may be particularly useful in the treatment of liver disorders which include, but are not limited to, viral diseases such as hepatitis B, hepatitis C or hepatitis D, liver cancer as well as other types of cancer involving metastases located in the liver.

The foregoing and other objects, features and advantages of the invention will be apparent from the description, drawings, and claims that follow.

### BRIEF DESCRIPTION OF THE DRAWINGS

FIGS. 1A-1C are schematic illustrations of non-limiting examples of fusion proteins constructed in accordance with the invention.

FIG. 2 is a graph showing the survival curves for groups of SCID mice injected with suspensions of Daudi cells and then treated with huFc-huIFN-alpha. On day 0, mice were injected with Daudi cells. On days 3-8, groups of eight mice were injected with PBS (diamonds), 30 μg of huFc-huIFN-alpha (crosses), or with 60 μg of huFc-huIFN-alpha (triangles).

FIG. 3 is a graph showing the growth rates of subcutaneous tumors of Daudi cells in SCID mice treated with huFc-huIFN-alpha. About four weeks prior to treatment, mice were subcutaneously injected with Daudi cells. When the injected Daudi cells had grown to form tumors of 200-400 mm³, mice were sorted in groups of eight and treated for six days with an injection of PBS (diamonds), 30 μg of huFc-huIFN-alpha in PBS (squares), or 60 μg of huFc-huIFN-alpha in PBS (triangles).

### DETAILED DESCRIPTION OF THE INVENTION

Many conditions may be alleviated by the administration of interferon-alpha. For example, as discussed previously, interferons alpha 2a and 2b (trade names Roferon and Intron A, respectively) are useful in the treatment of chronic hepatitis B, C and D, condylomata acuminata (genital warts), AIDS-related Kaposi's sarcoma, hairy cell leukemia, malignant melanoma, basal cell carcinoma, multiple myeloma, renal cell carcinoma, herpes I and II, varicella/herpes zoster, and mycosis fungoides. Furthermore, studies have been performed to evaluate the efficacy of interferon-alpha in the treatment of prostate cancer and chronic myelogenous leukemia.

For the treatment of hepatitis, for example, it can be particularly useful to have a form of interferon-alpha which is concentrated in the liver. In this way, the concentration of interferon-alpha in other tissues can be minimized, thereby reducing side effects. Liver tissue is the primary site for removal of soluble immune complexes, and Fc receptors are abundant on liver macrophages (Kupffer cells) (Benacerraf, B. et al. (1959) J. IMMUNOL. 82: 131; Paul, W. E. (1993) FUNDAMENTALS OF IMMUNOLOGY, 3rd ed. ch. 5:113-116). Accordingly, by fusing interferon-alpha to an immunoglobulin Fc region, the interteron-alpha molecule can be targeted preferably to liver tissue relative to the same interferon-alpha molecule lacking the immunoglobulin Fc region. The IgG type of antibody that has the highest affinity for the Fc receptors are IgG1. However, in contrast, IgG4, for example, has an approximately 10-fold lower affinity for the Fc gamma receptor I (Anderson and Abraham (1980) J. IMMUNOL. 125: 2735; Woof et al. (1986) MOL. IMMUNOL. 23: 319). Fc-gamma 1 from IgG1, when placed at the C-terminus of a ligand, can mediate antibody-dependent cell-mediated cytotoxicity (ADCC) against cells that express a receptor for that ligand. In addition, Fc-gamma 1, when present on the C-terminus of a ligand, can mediate C1q binding and complement fixation directed against cells expressing a receptor for that ligand.

In contrast to IgG1, IgG4 does not effectively fix complement. This has led to the proposal that an N-terminal interferon-alpha could be fused to a C-terminal Fc region from IgG4 (Chang, T. W. et al., U.S. Pat. No. 5,723,125). However, when the Fc region of IgG4 is separated from the Fab region, the Fc of IgG4 fixes complement as well as the Fc region of IgG1 (Isenman, D. E. et al. (1975) J. IMMUNOL. 114: 1726). Based on this result and the fact that the Fc sequences of IgG1 and IgG4 are quite similar, without wishing to be bound by theory, it is contemplated that the Fab region of IgG4 sterically blocks C1q binding and complement fixation because the hinge region connecting the IgG4 Fab and Fc regions is shorter than the hinge of IgG1. If the large, bulky Fab region of IgG4 is replaced by a small molecule, such as interferon-alpha, and the interferon-alpha and Fc region are connected by a flexible linker, it is contemplated that such an interferon-alpha-Fc-gamma 4 fusion would fix complement when bound to cells bearing interferon-alpha receptors.

The cytotoxic effect due to the fusion of an N-terminal cytokine and a C-terminal Fc region is well known. For example, fusion of the cytokine interleukin-2 (IL-2) to an Fc region creates a molecule that is able to fix complement and cause lysis of cells bearing the IL-2 receptor (Landolfi, N. F., U.S. Pat. No. 5,349,053).

Fusions in which an Fc region is placed at the N-terminus of a ligand (termed ‘immunofusins’ or ‘Fc-X’ fusions, where X is a ligand such as Interferon-alpha) have a number of distinctive, advantageous biological properties (Lo et al., U.S. Pat. Nos. 5,726,044 and 5,541,087; Lo et al. (1998) PROTEIN ENGINEERING 11: 495). In particular, such fusion proteins can still bind to the relevant Fc receptors on cell surfaces. However, when the ligand binds to its receptor on a cell surface, the orientation of the Fc region is altered and the sequences that mediate ADCC and complement fixation appear to be occluded. As a result, the Fc region in an Fc-X molecule does not mediate ADCC or complement fixation effectively. Thus, Fc-X fusions are expected to have the virtues of increased serum half-life and relative concentration in the liver, with little deleterious effects from ADCC and complement fixation.

One feature of the Fc-X constructs of the invention is to concentrate the target protein, in this case interferon-alpha, in the liver. The Fc region from the gamma1 and gamma3 chains show the highest affinity for the Fc receptor, with the gamma4 chain showing a reduced affinity and the gamma2 chain showing extremely low affinity to the Fc receptor. Accordingly, Fc regions derived from gamma1 or gamma3 chains preferably are used in the Fc-X constructs of the invention because they have the highest affinities for Fc receptors and thus can target the interferon-alpha preferentially to liver tissues. This is in contrast to an X-Fc protein, for example, an interferon-alpha-Fc fusion protein where the potential advantage of concentration in the liver must be balanced by the fact that this fusion protein can mediate effector functions, namely complement fixation and ADCC, directed against cells bearing receptors for interferon-alpha.

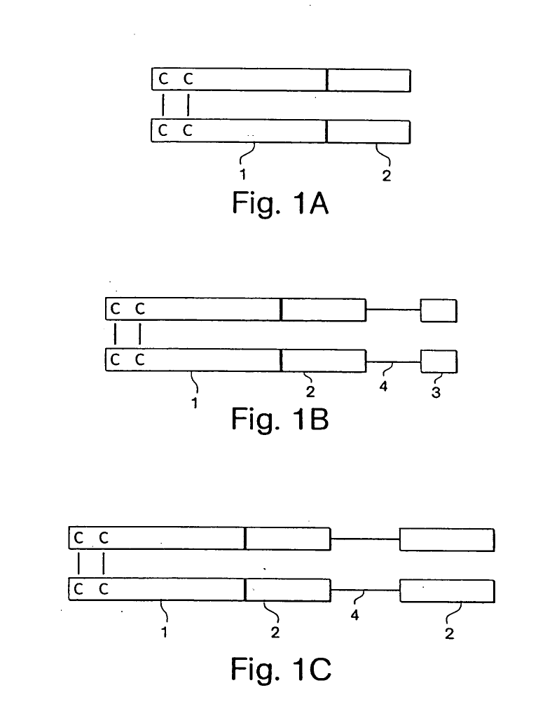

The invention thus provides nucleic acid sequences encoding and amino acid sequences defining fusion proteins comprising an immunoglobulin Fc region and at least one target protein, referred to herein as interferon-alpha. Three exemplary embodiments of protein constructs embodying the invention are illustrated in the drawing as FIGS. 1A-1C. Because dimeric constructs are preferred, all are illustrated as dimers cross-linked by a pair of disulfide bonds between cysteines in adjacent subunits. In the drawings, the disulfide bonds are depicted as linking together the two immunoglobulin heavy chain Fc regions via an immunoglobulin hinge region within each heavy chain, and thus are characteristic of native forms of these molecules. While constructs including the hinge region of Fc are preferred and have been shown promise as therapeutic agents, the invention contemplates that the crosslinking at other positions may be chosen as desired. Furthermore, under some circumstances, dimers or multimers useful in the practice of the invention may be produced by non-covalent association, for example, by hydrophobic interaction. Because homodimeric constructs are important embodiments of the invention, the drawings illustrate such constructs. It should be appreciated, however, that heterodimeric structures also are useful in the practice of the invention.

FIG. 1A illustrates a dimeric construct produced in accordance with the principles set forth herein (see, for example, Example 1). Each monomer of the homodimer comprises an immunoglobulin Fc region 1 including a hinge region, a CH2 domain and a CH3 domain. Attached directly, i.e., via a polypeptide bond, to the C terminus of the Fc region is interferon-alpha 2. It should be understood that the Fc region may be attached to a target protein via a polypeptide linker (not shown).

FIGS. 1B and 1C depict protein constructs of the invention which include as a target protein plural interferon-alpha proteins arranged in tandem and connected by a linker. In FIG. 1B, the target protein comprises full length interferon-alpha 2, a polypeptide linker made of glycine and serine residues 4, and an active variant of interferon-alpha 3. FIG. 1C differs from the construct of FIG. 1B in that the most C-terminal protein domain comprises a second, full length copy of interferon-alpha 2. Although FIGS. 1A-1C represent Fc-X constructs, where X is the target protein, it is contemplated that useful proteins of the invention may also be depicted by the formula X-Fc-X, wherein the X's may represent the same or different target proteins.

As used herein, the term “polypeptide linker” is understood to mean a polypeptide sequence that can link together two proteins that in nature are not naturally linked together. The polypeptide linker preferably comprises a plurality of amino acids such as alanine, glycine and serine or combinations of such amino acids. Preferably, the polypeptide linker comprises a series of glycine and serine peptides about 10-15 residues in length. See, for example, U.S. Pat. No. 5,258,698. It is contemplated, however, that the optimal linker length and amino acid composition may be determined by routine experimentation.

As used herein, the term “multivalent” refers to a recombinant molecule that incorporates two or more biologically active segments. The protein fragments forming the multivalent molecule optionally may be linked through a polypeptide linker which attaches the constituent parts and permits each to function independently.

As used herein, the term “bivalent” refers to a multivalent recombinant molecule having the configuration Fc-X or X-Fc, where X is a target molecule. The immunoglobulin Fc regions can associate, for example, via interchain disulfide bonds, to produce the type of constructs shown in FIGS. 1A. If the fusion construct of the invention has the configuration Fc-X-X, the resulting Fc molecule is shown in FIG. 1C. The two target proteins may be linked through a peptide linker. Constructs of the type shown in FIG. 1A can increase the apparent binding affinity between the target molecule and its receptor.

As used herein, the term “multimeric” refers to the stable association of two or more polypeptide chains either covalently, for example, by means of a covalent interaction, for example, a disulfide bond, or non-covalently, for example, by hydrophobic interaction. The term multimer is intended to encompass both homomultimers, wherein the subunits are the same, as well as, heteromultimers, wherein the subunits are different.

As used herein, the term “dimeric” refers to a specific multimeric molecule where two polypeptide chains are stably associated through covalent or non-covalent interactions. Such constructs are shown schematically in FIG. 1A. It should be understood that the immunoglobulin Fc region including at least a portion of the hinge region, a CH2 domain and a CH3 domain, typically forms a dimer. Many protein ligands are known to bind to their receptors as a dimer. If a protein ligand X dimerizes naturally, the X moiety in an Fc-X molecule will dimerize to a much greater extent, since the dimerization process is concentration dependent. The physical proximity of the two X moieties connected by Fc would make the dimerization an intramolecular process, greatly shifting the equilibrium in favor of the dimer and enhancing its binding to the receptor.

As used herein, the term “interferon-alpha” is understood to mean not only full length mature interferon-alpha, for example, human interferon-alpha 1 (SEQ ID NO: 8), human interferon-alpha 2 (SEQ ID NO: 9), human interferon-alpha 4 (SEQ ID NO: 10), human interferon-alpha 5 (SEQ ID NO: 11), human interferon-alpha 6 (SEQ ID NO: 12), human interferon-alpha 7 (SEQ ID NO: 13), human interferon-alpha 8 (SEQ ID NO: 14), human interferon-alpha 10 (SEQ ID NO: 15), human interferon-alpha 14 (SEQ ID NO: 16), human interferon-alpha 16 (SEQ ID NO: 17), human interferon-alpha 17 (SEQ ID NO: 18), human interferon-alpha 21 (SEQ ID NO: 19), interferon delta-1 (SEQ ID NO: 20), II-1 (interferon omega-1) (SEQ ID NO: 21); and mouse interferon-alpha 1 (SEQ ID NO: 22), mouse interferon-alpha 2 (SEQ ID NO: 23), mouse interferon-alpha 4 (SEQ ID NO: 24), mouse interferon-alpha 5 (SEQ ID NO: 25), mouse interferon-alpha 6 (SEQ ID NO: 26), mouse interferon-alpha 7 (SEQ ID NO: 27), mouse interferon-alpha 8 (SEQ ID NO 28), and mouse interferon-alpha 9 (SEQ ID NO: 29), but also variants and bioactive fragments thereof. Known sequences of interferon-alpha may be found in GenBank.

The term bioactive fragment refers to any interferon-alpha protein fragment that has at least 50%, more preferably at least 70%, and most preferably at least 90% of the biological activity of the template human interferon-alpha protein of SEQ ID NO: 2, as determined using the cell proliferation inhibition assay of Example 4. The term variants includes species and allelic variants, as well as other naturally occurring or non-naturally occurring variants, for example, generated by genetic engineering protocols, that are at least 70% similar or 60% identical, more preferably at least 75% similar or 65% identical, and most preferably at least 80% similar or 70% identical to the mature human interferon-alpha protein disclosed in SEQ ID NO.: 2.

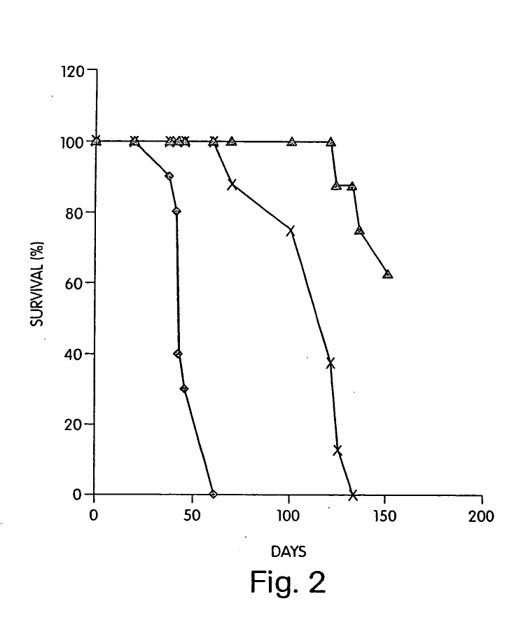

To determine whether a candidate polypeptide has the requisite percentage similarity or identity to a reference polypeptide, the candidate amino acid sequence and the reference amino acid sequence are first aligned using the dynamic programming algorithm described in Smith and Waterman (1981) J. MOL. BIOL. 147:195-197, in combination with the BLOSUM62 substitution matrix described in FIG. 2 of Henikoff and Henikoff (1992), “Amino acid substitution matrices from protein blocks”, PROC. NATL. ACAD. SCI. USA 89:10915-10919. For the present invention, an appropriate value for the gap insertion penalty is −12, and an appropriate value for the gap extension penalty is −4. Computer programs performing alignments using the algorithm of Smith-Waterman and the BLOSUM62 matrix, such as the GCG program suite (Oxford Molecular Group, Oxford, England), are commercially available and widely used by those skilled in the art.

Once the alignment between the candidate and reference sequence is made, a percent similarity score may be calculated. The individual amino acids of each sequence are compared sequentially according to their similarity to each other. If the value in the BLOSUM62 matrix corresponding to the two aligned amino acids is zero or a negative number, the pair-wise similarity score is zero; otherwise the pair-wise similarity score is 1.0. The raw similarity score is the sum of the pair-wise similarity scores of the aligned amino acids. The raw score then is normalized by dividing it by the number of amino acids in the smaller of the candidate or reference sequences. The normalized raw score is the percent similarity. Alternatively, to calculate a percent identity, the aligned amino acids of each sequence again are compared sequentially. If the amino acids are non-identical, the pair-wise identity score is zero; otherwise the pair-wise identity score is 1.0. The raw identity score is the sum of the identical aligned amino acids. The raw score is then normalized by dividing it by the number of amino acids in the smaller of the candidate or reference sequences. The normalized raw score is the percent identity. Insertions and deletions are ignored for the purposes of calculating percent similarity and identity. Accordingly, gap penalties are not used in this calculation, although they are used in the initial alignment.

Variants may also include other interferon-alpha mutant proteins having interferon-alpha-like activity. Species and allelic variants, include, but are not limited to human and mouse interferon-alpha sequences. Human interferon-alpha variants are shown in SEQ ID NOS: 8-21, and mouse interferon-alpha variants are shown in SEQ ID NOS: 22-29.

Furthermore, the interferon-alpha sequence may comprise a portion or all of the consensus sequence set forth in SEQ ID NO: 7, wherein the interferon-alpha has at least 50%, more preferably at least 70%, and most preferably at least 90% of the biological activity of the mature human interferon-alpha of SEQ ID NO: 2, as determined using the cell proliferation inhibition assay of Example 4.

These proteins have very similar purification properties and other biological properties. In particular, the DNA manipulation, fusion protein expression, and fusion protein purification properties of Fc-Interferon-alpha proteins are extremely similar. For example, human interferon-alpha 2a and human interferon-alpha 2b differ by one amino acid only, whereas the interferon-alpha 2a has a lysine residue at the same position that interferon-alpha 2b has an arginine residue. Human interferon-alpha 2a and human interferon-alpha 2b have extremely similar properties and are interchangeable for all known purposes.

The three-dimensional structure of interferon-alpha has been solved by X-ray crystallography (Ramaswamy et al. (1986) STRUCTURE 4: 1453). The sequences of interferon-alpha proteins are so similar that the determined structure is regarded as a structure for the entire family of proteins. The three-dimensional structure of interferon-alpha, like that of interferon-beta, is a dimer with a zinc ion at the dimer interface. However, in solution, interferon-alpha behaves as a monomer. It has been proposed, by analogy with the cytokine IL-6 and other protein ligands, that interferon-alpha may dimerize upon receptor binding (Radhakrishnan, R. et al. (1996) STRUCTURE 4: 1453; Karpusas, M. et al. (1997) PROC. NAT. ACAD. SCI. USA 94: 11813).

Dimerization of a ligand can increase the apparent binding affinity between the ligand and its receptor. For instance, if one interferon-alpha moiety of an Fc-Interferon-alpha fusion protein can bind to a receptor on a cell with a certain affinity, the second interferon-alpha moiety of the same Fc-Interferon-alpha fusion protein may bind to a second receptor on the same cell with a much higher avidity (apparent affinity). This may occur because of the physical proximity of the second interferon-alpha moiety to the receptor after the first interferon-alpha moiety already is bound. In the case of an antibody binding to an antigen, the apparent affinity may be increased by at least ten thousand-fold, i.e., 10⁴. Each protein subunit, i.e., “X,” has its own independent function so that in a multivalent molecule, the functions of the protein subunits may be additive or synergistic. Thus, fusion of the normally dimeric Fc molecule to interferon-alpha may increase the activity of interferon-alpha. Accordingly, constructs of the type shown in FIG. 1A may increase the apparent binding affinity between interferon-alpha and its receptor.

The target proteins disclosed herein are expressed as fusion proteins with an Fc region of an immunoglobulin. As is known, each immunoglobulin heavy chain constant region comprises four or five domains. The domains are named sequentially as follows: CH1-hinge-CH2—CH3(—CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE.

As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain.

The currently preferred class of immunoglobulin from which the heavy chain constant region is derived is IgG (Igγ) (γ subclasses 1, 2, 3, or 4). The nucleotide and amino acid sequences of human Fc γ-1 are set forth in SEQ ID NOS: 3 and 4. Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fcγ or the homologous domains in any of IgA, IgD, IgE, or IgM.

Depending on the application, constant region genes from species other than human, for example, mouse or rat may be used. The immunoglobulin Fc region used as a fusion partner in the DNA construct generally may be from any mammalian species. Where it is undesirable to elicit an immune response in the host cell or animal against the Fc region, the Fc region may be derived from the same species as the host cell or animal. For example, a human immunoglobulin Fc region can be used when the host animal or cell is human; likewise, a murine immunoglobulin Fc region can be used where the host animal or cell will be a mouse.

Nucleic acid sequences encoding, and amino acid sequences defining a human immunoglobulin Fc region useful in the practice of the invention are set forth in SEQ ID NOS: 3 and 4. However, it is contemplated that other immunoglobulin Fc region sequences useful in the practice of the invention may be found, for example, by those encoded by nucleotide sequences disclosed in the Genbank and/or EMBL databases, for example, AF045536.1 (Macaca fuscicularis), AF045537.1 (Macaca mulatta), AB016710 (Felix catus), K00752 (Oryctolagus cuniculus), U03780 (Sus scrofa), Z48947 (Camelus dromedarius), X62916 (Bos taurus), L07789 (Mustela vison), X69797 (Ovis aries), U17166 (Cricetulus migratorius), X07189 (Rattus rattus), AF57619.1 (Trichosurus vulpecula), or AF035195 (Monodelphis domestica), the disclosures of which are incorporated by reference herein.

Furthermore, it is contemplated that substitution or deletion of amino acids within the immunoglobulin heavy chain constant regions may be useful in the practice of the invention. One example may include introducing amino acid substitutions in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

The use of human Fcγ1 as the Fc region sequence has several advantages. For example, if the Fc fusion protein is to be used as a biopharmaceutical, the Fcγ1 domain may confer effector function activities to the fusion protein. The effector function activities include the biological activities such as placental transfer and increased serum half-life. The immunoglobulin Fc region also provides for detection by anti-Fc ELISA and purification through binding to Staphylococcus aureus protein A (“Protein A”). In certain applications, however, it may be desirable to delete specific effector functions from the immunoglobulin Fc region, such as Fc receptor binding and/or complement fixation.

It is understood that the present invention exploits conventional recombinant DNA methodologies for generating the Fc fusion proteins useful in the practice of the invention. The Fc fusion constructs preferably are generated at the DNA level, and the resulting DNAs integrated into expression vectors, and expressed to produce the fusion proteins of the invention. As used herein, the term “vector” is understood to mean any nucleic acid comprising a nucleotide sequence competent to be incorporated into a host cell and to be recombined with and integrated into the host cell genome, or to replicate autonomously as an episome. Such vectors include linear nucleic acids, plasmids, phagemids, cosmids, RNA vectors, viral vectors and the like. Non-limiting examples of a viral vector include a retrovirus, an adenovirus and an adeno-associated virus. As used herein, the term “gene expression” or “expression” of a target protein, is understood to mean the transcription of a DNA sequence, translation of the mRNA transcript, and secretion of an Fc fusion protein product.

A useful expression vector is pdCs (Lo et al. (1988) PROTEIN ENGINEERING 11:495, in which the transcription of the Fc-X gene utilizes the enhancer/promoter of the human cytomegalovirus and the SV40 polyadenylation signal. The enhancer and promoter sequence of the human cytomegalovirus used was derived from nucleotides −601 to +7 of the sequence provided in Boshart et al. (1985) CELL 41:521. The vector also contains the mutant dihydrofolate reductase gene as a selection marker (Simonsen and Levinson (1983) PROC. NAT. ACAD. SCI. USA 80:2495).

An appropriate host cell can be transformed or transfected with the DNA sequence of the invention, and utilized for the expression and/or secretion of the target protein. Currently preferred host cells for use in the invention include immortal hybridoma cells, NS/O myeloma cells, 293 cells, Chinese hamster ovary cells, HELA cells, and COS cells.

One expression system that has been used to produce high level expression of fusion proteins in mammalian cells is a DNA construct encoding, in the 5′ to 3′ direction, a secretion cassette, including a signal sequence and an immunoglobulin Fc region, and a target protein. Several target proteins have been expressed successfully in such a system and include, for example, IL2, CD26, Tat, Rev, OSF-2, βIG-H3, IgE Receptor, PSMA, and gp120. These expression constructs are disclosed in U.S. Pat. Nos. 5,541,087 and 5,726,044 to Lo et al.

As used herein, the term “signal sequence” is understood to mean a segment which directs the secretion of the interferon-alpha fusion protein and thereafter is cleaved following translation in the host cell. The signal sequence of the invention is a polynucleotide which encodes an amino acid sequence which initiates transport of a protein across the membrane of the endoplasmic reticulum. Signal sequences which are useful in the invention include antibody light chain signal sequences, e.g., antibody 14.18 (Gillies et. al. (1989) J. IMMUNOL. METH. 125:191), antibody heavy chain signal sequences, e.g., the MOPC141 antibody heavy chain signal sequence (Sakano et al. (1980) NATURE 286:5774), and any other signal sequences which are known in the art (see, e.g., Watson (1984) NUCLEIC ACIDS RESEARCH 12:5145).

Signal sequences have been well characterized in the art and are known typically to contain 16 to 30 amino acid residues, and may contain greater or fewer amino acid residues. A typical signal peptide consists of three regions: a basic N-terminal region, a central hydrophobic region, and a more polar C-terminal region. The central hydrophobic region contains 4 to 12 hydrophobic residues that anchor the signal peptide across the membrane lipid bilayer during transport of the nascent polypeptide. Following initiation, the signal peptide is usually cleaved within the lumen of the endoplasmic reticulum by cellular enzymes known as signal peptidases. Potential cleavage sites of the signal peptide generally follow the “(−3, −1) rule”. Thus a typical signal peptide has small, neutral amino acid residues in positions −1 and −3 and lacks proline residues in this region. The signal peptidase will cleave such a signal peptide between the −1 and +1 amino acids. Thus, the signal sequence may be cleaved from the amino-terminus of the fusion protein during secretion. This results in the secretion of an Fc fusion protein consisting of the immunoglobulin Fc region and the target protein. A detailed discussion of signal peptide sequences is provided by von Heijne (1986) NUCLEIC ACIDS RES. 14:4683.

As would be apparent to one of skill in the art, the suitability of a particular signal sequence for use in the secretion cassette may require some routine experimentation. Such experimentation will include determining the ability of the signal sequence to direct the secretion of an Fc fusion protein and also a determination of the optimal configuration, genomic or cDNA, of the sequence to be used in order to achieve efficient secretion of Fc fusion proteins. Additionally, one skilled in the art is capable of creating a synthetic signal peptide following the rules presented by von Heijne, referenced above. and testing for the efficacy of such a synthetic signal sequence by routine experimentation. A signal sequence can also be referred to as a “signal peptide,” “leader sequence,” or “leader peptides.”

The fusion of the signal sequence and the immunoglobulin Fc region is sometimes referred to herein as secretion cassette. An exemplary secretion cassette useful in the practice of the invention is a polynucleotide encoding, in a 5′ to 3′ direction, a signal sequence of an immunoglobulin light chain gene and an Fcγ1 region of the human immunoglobulin γ1 gene. The Fcγ1 region of the immunoglobulin Fcγ1 gene preferably includes at least a portion of the immunoglobulin hinge domain and at least the CH3 domain, or more preferably at least a portion of the hinge domain, the CH2 domain and the CH3 domain. As used herein, the “portion” of the immunoglobulin hinge region is understood to mean a portion of the immunoglobulin hinge that contains at least one, preferably two cysteine residues capable of forming interchain disulfide bonds. The DNA encoding the secretion cassette can be in its genomic configuration or its cDNA configuration. Under certain circumstances, it may be advantageous to produce the Fc region from human immunoglobulin Fcγ2 heavy chain sequences. Although Fc fusions based on human immunoglobulin γ1 and γ2 sequences behave similarly in mice, the Fc fusions based on the γ2 sequences can display superior pharmacokinetics in humans.

In another embodiment, the DNA sequence encodes a proteolytic cleavage site interposed between the secretion cassette and the target protein. A cleavage site provides for the proteolytic cleavage of the encoded fusion protein thus separating the Fc domain from the target protein. As used herein, “proteolytic cleavage site” is understood to mean amino acid sequences which are preferentially cleaved by a proteolytic enzyme or other proteolytic cleavage agents. Useful proteolytic cleavage sites include amino acids sequences which are recognized by proteolytic enzymes such as trypsin, plasmin or enterokinase K. Many cleavage site/cleavage agent pairs are known (see, for example, U.S. Pat. No. 5,726,044).

Further, substitution or deletion of constructs of these constant regions, in which one or more amino acid residues of the constant region domains are substituted or deleted also would be useful. One example would be to introduce amino acid substitutions in the upper CH2 region to create an Fc variant with reduced affinity for Fc receptors (Cole et al. (1997) J. IMMUNOL. 159: 3613). One of ordinary skill in the art can prepare such constructs using well known molecular biology techniques.

In the Examples disclosed herein, high levels of Fc-Interferon-alpha were produced. The initial clones produced about 50 μg/mL of Fc-Interferon-alpha, which could be purified readily to homogeneity by Protein A affinity chromatography. Expression levels often can be increased several fold by subcloning. As stated above, it is found that when interferon-alpha is expressed as Fc fusion molecules, high levels of expression are obtained, presumably because the Fc portion acts as a carrier, helping the polypeptide at the C-terminus to fold correctly and to be secreted efficiently. Moreover, the Fc region is glycosylated and highly charged at physiological pH, thus the Fc region can help to solubilize hydrophobic proteins.

In addition to the high levels of expression, interferon-alpha fusion proteins exhibited longer serum half-lives compared to interferon-alpha alone, due in part to their larger molecular sizes. For example, Fc-Interferon-alpha has a circulating half-life of 19.3 hours in mouse (see Example 6), as compared to 2-5 hours for interferon-alpha (PHYSICIANS DESK REFERENCE, 50th edition, 1996:2156-2147 and 2364-2373). Interferon-alpha, having a molecular weight of about 19 kD, is small enough to be cleared efficiently by renal filtration. In contrast, Fc-Interferon-alpha has a molecular weight of about 100 kD since there are two interferon-alpha moieties attached to each Fc molecule (i.e., two interferon-alphas since Fc is in its dimeric form). Such a dimeric structure may exhibit a higher binding affinity to the interferon-alpha receptor. Since the interferon-alpha activity is receptor-mediated, the bivalent interferon-alpha fusion proteins will be potentially more efficacious than interferon-alpha itself.

Additionally, many protein ligands are known to bind to their receptors as a dimer. Since interferon-alpha belongs to a class of protein ligands with weak dimerization constants, the physical constraint imposed by the Fc on interferon-alpha would make the dimerization an intramolecular process, thus, shifting the equilibrium in favor of the dimer and enhancing its binding to the receptors. Cysteine residues also can be introduced by standard recombinant DNA technology to the monomer at appropriate places to stabilize the dimer through covalent disulfide bond formation.

The fusion proteins of the invention provide several important clinical benefits. As demonstrated in the tests of biological activity in the Daudi cell and cytopathic effect assays (Example 4), the biological activity of Fc-Interferon-alpha is significantly higher than that of interferon-alpha.

Another embodiment of the present invention provides constructs having various structural conformations, e.g., bivalent or multivalent constructs, dimeric or multimeric constructs, and combinations thereof. Such functional conformations of molecules of the invention allow the synergistic effect of interferon-alpha and other anti-viral and anti-cancer proteins to be explored in animal models.

An important aspect of the invention is that the sequences and properties of various interferon-alpha proteins and encoding DNAs are quite similar. In the context of Fc-X fusions, the properties of interferon-alpha proteins and encoding DNAs are essentially identical, so that a common set of techniques can be used to generate any Fc-Interferon-alpha DNA fusion, to express the fusion, to purify the fusion protein, and to administer the fusion protein for therapeutic purposes.

The present invention also provides methods for the production of interferon-alpha of non-human species as Fc fusion proteins. Non-human interferon-alpha fusion proteins are useful for preclinical studies of interferon-alpha because efficacy and toxicity studies of a protein drug must be performed in animal model systems before testing in human beings. A human protein may not work in a mouse model since the protein may elicit an immune response, and/or exhibit different pharmacokinetics skewing the test results. Therefore, the equivalent mouse protein is the best surrogate for the human protein for testing in a mouse model.

The present invention provides methods of treating various cancers, viral diseases, other diseases, related conditions and causes thereof by administering the DNA, RNA or proteins of the invention to a mammal having such condition. Related conditions may include, but are not limited to, hepatitis B, hepatitis C, hepatitis D, genital warts, hairy-cell leukemia, AIDS-related Kaposi's sarcoma, melanoma, prostate cancer and other forms of viral disease and cancer. In view of the broad roles played by interferon-alpha in modulating immune responses, the present invention also provides methods for treating conditions alleviated by the administration of interferon-alpha. These methods include administering to a mammal having the condition, which may or may not be directly related to viral infection or cancer, an effective amount of a composition of the invention.

The proteins of the invention not only are useful as therapeutic agents, but one skilled in the art recognizes that the proteins are useful in the production of antibodies for diagnostic use. Likewise, appropriate administration of the DNA or RNA, e.g., in a vector or other delivery system for such uses, is included in methods of use of the invention.

As a fusion protein with the immunoglobulin Fc, Fc-Interferon-alpha may have a very favorable tissue distribution and a slightly different mode of action to achieve clinical efficacy, especially in view of its long serum half-life and the high dose of soluble protein that can be administered. In particular, there is a high level of Fc gamma receptor in the liver, which is the site of infection by the viruses causing hepatitis B and hepatitis D. Neurological side effects of interferon-alpha are thought to occur because the small size of interferon-alpha allows it to cross the blood-brain barrier. The much larger size of Fc-Interferon-alpha significantly reduces the extent to which this protein crosses the blood-brain barrier.

Compositions of the present invention may be administered by any route which is compatible with the particular molecules. It is contemplated that the compositions of the present invention may be provided to an animal by any suitable means, directly (e.g., locally, as by injection, implantation or topical administration to a tissue locus) or systemically (e.g., parenterally or orally). Where the composition is to be provided parenterally, such as by intravenous, subcutaneous, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the composition preferably comprises part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance. The fluid medium for the agent thus can comprise normal physiologic saline.

The DNA constructs (or gene constructs) of the invention also can be used as a part of a gene therapy protocol to deliver nucleic acids encoding interferon-alpha or a fusion protein construct thereof. The invention features expression vectors for in vivo transfection and expression of interferon-alpha or a fusion protein construct thereof in particular cell types so as to reconstitute or supplement the function of interferon-alpha. Expression constructs of interferon-alpha, or fusion protein constructs thereof, may be administered in any biologically effective carrier, e.g. any formulation or composition capable of effectively delivering the interferon-alpha gene or fusion protein construct thereof to cells in vivo. Approaches include insertion of the subject gene in viral vectors including recombinant retroviruses, adenovirus, adeno-associated virus, and herpes simplex virus-1, or recombinant bacterial or eukaryotic plasmids. Preferred dosages per administration of nucleic acids encoding the fusion proteins of the invention are within the range of 1 μg/m² to 100 mg/m², more preferably 20 μg/m² to 10 mg/m², and most preferably 400 μg/m² to 4 mg/m². It is contemplated that the optimal dosage and mode of administration may be determined by routine experimentation well within the level of skill in the art.

Preferred dosages of the fusion protein per administration are within the range of 0.1 mg/m²-100 mg/m², more preferably, 1 mg/m²-20 mg/m², and most preferably 2 mg/m²-6 mg/m². It is contemplated that the optimal dosage, however, also depends upon the disease being treated and upon the existence of side effects. However, optimal dosages may be determined using routine experimentation. Administration of the fusion protein may be by periodic bolus injections, or by continuous intravenous or intraperitoneal administration from an external reservoir (for example, from an intravenous bag) or internal (for example, from a bioerodable implant). Furthermore, it is contemplated that the fusion proteins of the invention also may be administered to the intended recipient together with a plurality of different biologically active molecules. It is contemplated, however, that the optimal combination of fusion protein and other molecules, modes of administration, dosages may be determined by routine experimentation well within the level of skill in the art.

The invention is illustrated further by the following non-limiting examples.

### EXAMPLES

### Example 1

### Expression of huFc-huInterferon-alpha (huFc-IFN-alpha)

mRNA was prepared from human peripheral blood mononuclear cells and reverse transcribed with reverse transcriptase. The resultant cDNA was used as template for Polymerase Chain Reactions (PCR) to clone and adapt the human interferon-alpha cDNA for expression as a huFc-Interferon-alpha (huFc-IFN-alpha) fusion protein. The forward primer was 5′ C CCG GGT AAA TGT GAT CTG CCT CAG AC (SEQ ID NO: 5), where the sequence CCCGGG (XmaI restriction site) TAAA encodes the carboxy terminus of the immunoglobulin heavy chain, followed by sequence (in bold) encoding the N-terminus of interferon-alpha. The reverse primer was 5′ CTC GAG TCA ATC CTT CCT CCT TAA TC (SEQ ID NO: 6), which encodes the carboxy-terminal sequence (anti-sense) of interferon-alpha with its translation STOP codon (anticodon, TCA), and this was followed by an XhoI site (CTCGAG). A 517 base-pair PCR product was cloned and sequenced. Sequence analysis confirmed that the PCR product encodes mature human Interferon-alpha adapted for expression, i.e., with a XmaI at the 5′ end and a XhoI site at the 3′ end.

The expression vector pdCs-huFc-IFN-alpha was constructed as follows. The XmaI-XhoI restriction fragment containing the human interferon-alpha cDNA was ligated to the XmaI-XhoI fragment of the pdCs-huFc vector according to Lo et al. (1998) Protein Engineering 11: 495. huFc is the human Fc fragment of the human immunoglobulin gamma 1. The resultant vector, pdCs-huFc-IFN-alpha, was used to transfect mammalian cells for the expression of huFc-IFN-alpha.

### Example 2

### Transfection and Expression of Protein

For transient transfection, the plasmid pdCs-huFc-IFN-alpha was introduced into human kidney 293 cells by coprecipitation of plasmid DNA with calcium phosphate (Sambrook et al. eds. (1989) “MOLECULAR CLONING—A LABORATORY MANUAL,” Cold Spring Harbor Press, NY) or by lipofection using Lipofectamine Plus (Life Technologies, Gaithersburg, Md.) in accordance with the manufacturer's instructions.

In order to obtain stably transfected clones, plasmid DNA was introduced into mouse myeloma NS/0 cells by electroporation. Briefly, NS/0 cells were grown in Dulbecco's modified Eagle's medium supplemented with 10% fetal bovine serum, 2 mM glutamine and penicillin/streptomycin. About 5×10⁶ cells were washed once with phosphate buffered saline (PBS) and resuspended in 0.5 mL PBS. Ten μg of linearized plasmid DNA then was incubated with the cells in a Gene Pulser Cuvette (0.4 cm electrode gap, BioRad) on ice for 10 min. Electroporation was performed using a Gene Pulser (BioRad, Hercules, Calif.) with settings at 0.25 V and 500 μF. Cells were allowed to recover for 10 min. on ice, after which they were resuspended in growth medium and then plated onto two 96 well plates. Stably transfected clones were selected by growth in the presence of 100 nM methotrexate (MTX), which was introduced two days post-transfection. The cells were fed every 3 days for two to three more times, and MTX-resistant clones appeared in 2 to 3 weeks. Supernatants from clones were assayed by anti-Fc ELISA (see Example 3) to identify high producers. High producing clones were isolated and propagated in growth medium containing 100 nM MTX.

For routine characterization by gel electrophoresis, Fc fusion proteins in the conditioned media were bound to Protein A Sepharose (Repligen, Cambridge, Mass.) and then eluted from the Protein A Sepharose by boiling in a standard protein sample buffer with or without 2-mercaptoethanol. After electrophoresis on a sodium dodecyl sulfate-polyacrylamide gel electrophoresis (SDS-PAGE), the protein bands were visualized by staining with Coomassie blue. By SDS-PAGE, the huFc-huInterferon-alpha had an apparent MW of about 52 kD.

For purification, the fusion proteins bound on Protein A Sepharose were eluted in a sodium phosphate buffer (100 mM NaH₂PO₄, pH 3, and 150 mM NaCl). The eluate then was immediately neutralized with 0.1 volume of 2 M Tris-hydrochoride, pH 8.

### Example 3

### ELISA Procedures

The concentration of human Fc-containing protein products in the supernatants of MTX-resistant clones and other test samples were determined by anti-huFc ELISA. The procedures are described in detail below.

A. Coating Plates.

ELISA plates were coated with AffiniPure Goat anti-Human IgG (H+L) (Jackson Immuno Research Laboratories, West Grove, Pa.) at 5 μg/mL in PBS, and 100 μL/well in 96-well plates (Nunc-Immuno plate Maxisorp). Coated plates were covered and incubated at 4° C. overnight. Plates then were washed 4 times with 0.05% Tween (Tween 20) in PBS, and blocked with 1% BSA/1% goat serum in PBS, 200 μL/well. After incubation with the blocking buffer at 37° C. for 2 hrs, the plates were washed 4 times with 0.05% Tween in PBS and tapped dry on paper towels.

B. Incubation with Test Samples and Secondary Antibody

Test samples were diluted as appropriate in sample buffer (1% BSA/1% goat serum/0.05% Tween in PBS). A standard curve was prepared using a chimeric antibody (with a human Fc), the concentration of which was known. To prepare a standard curve, serial dilutions were made in the sample buffer to give a standard curve ranging from 125 ng/mL to 3.9 ng/mL. The diluted samples and standards were added to the plate, 100 μL/well and the plate incubated at 37° C. for 2 hr. After incubation, the plate was washed 8 times with 0.05% Tween in PBS. To each well was then added 100 μL of the secondary antibody, the horseradish peroxidase-conjugated anti-human IgG (Jackson Immuno Research), diluted around 1:120,000 in the sample buffer. The exact dilution of the secondary antibody has to be determined for each lot of the HRP-conjugated anti-human IgG. After incubation at 37° C. for 2 hr, the plate was washed 8 times with 0.05% Tween in PBS.

C. Development

The substrate solution was added to the plate at 100 μL/well. The substrate solution was prepared by dissolving 30 mg of OPD (o-phenylenediamine dihydrochloride (OPD), (1 tablet) into 15 mL of 0.025 M Citric acid/0.05 M Na₂HPO₄ buffer, pH 5, which contained 0.03% of freshly added hydrogen peroxide. The color was allowed to develop for 30 min. at room temperature in the dark. The developing time is subject to change, depending on lot to lot variability of the coated plates, the secondary antibody, etc. The reaction was stopped by adding 4N sulfuric acid, 100 μL/well. The plate was read by a plate reader, which was set at both 490 and 650 nm and programmed to subtract the background OD at 650 nm from the OD at 490 nm.

### Example 4

### Bioassays

The bioactivity of huFc-huIFN-alpha was compared to that of human interferon-alpha (hu-IFN-alpha) human leucocyte interferon from Sigma, St. Louis, Mo.) using two different assays. The first assay determines the inhibition of proliferation of Daudi human lymphoblastoid B cell line (ATCC CCL 213). The second assay measures the inhibition of cytopathic effect of encephalomyocarditis virus (EMCV) on human lung carcinoma A549 cell line (ATCC CCL 185).

Interferon-alpha inhibits the proliferation of Daudi (human Burkett lymphoma) cells. Daudi cells were washed with serum-free RPMI 1640 twice, and resuspended in growth medium consisting of RPMI 1640 and 20% heat-inactivated (56° C.) fetal bovine serum. The cells then were plated at 1×10⁵ cells/mL/well on a 24-well plate in the presence of different concentrations of αIFH (2.1×10⁶ International units/mg) and huFc-huIFN-alpha. After 3-4 days, it was found that 50 pg/mL of IFN-alpha in the form of huFc-huIFN-alpha was as effective as 750 pg/mL of huIFN-alpha in achieving 50-100% inhibition of growth of Daudi cells. As a control, interferon-gamma (Pharmingen, San Diego, Calif.) at 100 ng/mL showed no activity in this assay. This demonstrates that the inhibition is interferon-alpha specific.

### Example 5

### Measurement of Antiviral Activity

Viral replication in cell culture often results in cytotoxicity, an effect known as cytopathic effect (CPE). Interferons can induce an antiviral state in cell cultures and protect cells from such CPE. The antiviral activity IFN-alpha can be quantitated by cytopathic effect reduction (CPER) assays, as described in “Lymphokines and Interferons: A Practical Approach,” edited by M. J. Clemens, A. G. Morris and A. J. H. Gearing, I.R.L. Press, Oxford, 1987. The antiviral activities of huFc-huIFN-alpha and huIFN-alpha were compared using the human lung carcinoma cell line A549 (ATCC CCL 185) and encephalmyocarditis virus (ATCC VR 129B) according to the CPER protocol described in the above reference. The effective doses to give 50% CPER (i.e., 50% protection) were found to be 570 pg/mL (based on the amount of IFN-alpha) for huFc-huIFN-alpha and 500 pg/mL for huIFN-alpha. Accordingly, the IFN-alpha in huFc-huIFN-alpha and huIFN-alpha have substantially equivalent anti-viral activity.

### Example 6

### Pharmacokinetics

The pharmacokinetics of huFc-huIFN-alpha was determined in a group of 4 Balb/c mice. Twenty-five milligrams of huFc-huIFN-alpha was injected into the tail vein of each mouse. Blood was obtained by retro-orbital bleeding immediately after injection (i.e., at t=0 min), and at 0.5, 1, 2, 4, 8 and 24 hr post injection. Blood samples were collected in tubes containing heparin to prevent clotting. Cells were removed by centrifugation in an Eppendorf high-speed microcentrifuge for 4 min. The concentration of huFc-huIFN-alpha in the plasma was measured by anti-huFc ELISA and Western blot analysis with anti-huFc antibody, which also showed that the huFc-huIFN-alpha stayed intact in circulation (52 kD band for huFc-huIFN-alpha). No degradation product (32 kD band for huFc) could be detected. The circulating half-life of huFc-huIFN-alpha was determined to be 19.3 hr, which is significantly longer than the reported circulating half-life of human IFN-alpha of about 2 to 5 hr (PHYSICIANS DESK REFERENCE, 50th edition, 1996:2145-2147 and 2364-2373).

### Example 7

### Treatment of Disseminated Growth of Human Burkitt Lymphoma in SCID Mice

Daudi (human Burkitt lymphoma) cells were grown in the C.B-17 SCID (Severe Combined Immune Deficiency) mice as disseminated tumors (Ghetie et al. (1990) INTL. J. CANCER: 45:481). About 5×10⁶ Daudi cells of a single cells suspension in 0.2 mL PBSB were injected intravenously into 6-8 week old SCID mice. Three days later, mice were randomized into three groups of eight and received daily intraperitoneal injections of 0.2 mL of PBS, 30 μg of huFc-huIFN-alpha (containing about 12 μg of IFN-alpha) in PBS, or 60 μg of huFc-huIFN-alpha in PBS. Mice were monitored daily. The results are presented in FIG. 2.

By Day 28 after the Daudi cell injection, all mice in the control PBS (diamonds) group had developed paralysis of the hind legs. Mice in this PBS control group began dying on Day 38 and by Day 61, all the mice in the control group died. In contrast, the mice in the treatment groups survived much longer, and in a dose-dependent manner. For the group that received 30 μg of huFc-huIFN-alpha (crosses), the first death occurred on Day 70, and all mice died by Day 134. Four the group that received 60 μg of huFc-huIFN-alpha (triangles), the first death did not occur till Day 126, and four more died on Day 153. The rest of the mice were sick and were euthanized.

### Example 8

### Treatment of Localized Growth of Human Burkett Lymphoma in SCID Mice

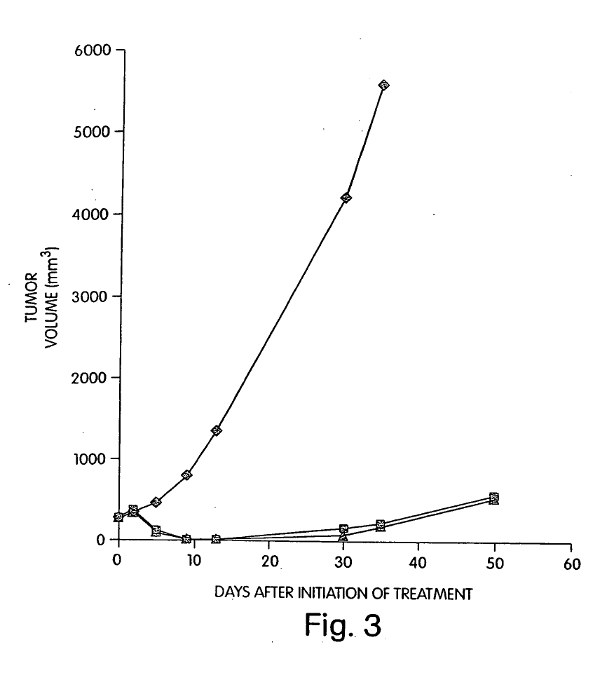

In this model, Daudi cells were grown in the C.B-17 SCID mice as subcutaneous tumors (Ghetie et al. (1990) INT. J. CANCER: 45-481). About 6×10⁶ Daudi cells of a single cell suspension in 0.1 mL PBS were injected subcutaneously into 6-8 week old SCID mice. Treatment started when the tumor size reached 200-400 mm³, which took about 4 weeks. Mice were randomized into 3 groups of 8, and each groups received 6 daily intraperitoneal injections of 0.2 mL of PBS, 30 μg of huFc-huIFN-alpha in PBS, or 60 μg of huFc-huIFN-alpha in PBS. The results are shown in FIG. 3. Size of tumors was measured twice a week.

The tumors in the control group mice (diamonds) grew rapidly to a mean volume of 5602 mm³ (range: 4343-6566 mm³) by day 35, after which all the mice in the group were euthanized. In contrast, the growth of tumors in the mice in the treatment groups were suppressed in a dose-dependent manner. The groups that received 30 μg and 60 μg of huFc-huIFN-alpha had mean tumor volumes of 214 and 170 mm³, respectively, at day 35, which were smaller than the 268 and 267 mm³ before treatment. In fact, the subcutaneous tumors had completely shrunk in 5 out of 8 mice in the group receiving 30 μg huFc-huIFN-alpha, and 4 out of 8 mice in the group receiving 60 μg of huFc-huIFN-alpha. Without further treatment, however, some of the tumors did return and grew. Nevertheless, two mice in the group remained tumor-free until day 205, when the experiment was terminated.

### Example 9

### Treatment of Liver Disease with Fc-Interferon-alpha

It is contemplated that a liver disease, for example, hepatitis or liver metastases, can be treated more effectively with Fc-Interferon-alpha than with interferon-alpha or interferon-alpha-Fc.

For example, it is contemplated that Fc-interferon-alpha can be effective in treating a mouse model in which tumor cells metastasize to the liver. Mice are anaesthetized by intraperitoneal injection of 80 mg/kg ketamine and 5 mg/kg xylazine in 0.2 ml PBS about 5 minutes before surgery. The following steps then are performed in a laminar flow hood to ensure sterility. The skin of each mouse is cleaned with betadine and ethanol Tumor cells, such as Daudi cells, are injected in 100 microliters of RPMI 1640 medium without supplement beneath the splenic capsule over a period of about one minute using a 27-gauge needle. After two minutes, the splenic pedicle is ligated with a 4.0 silk suture and the spleen is removed.

Some cells are carried from the site of injection into the liver, where they can form metastatic tumors. Mice with metastatic liver tumors then are treated with Fc-interferon-alpha. It is contemplated that mice treated with Fc-interferon-alpha show a significant reduction in tumor growth relative to mice treated with an equimolar amount of interferon-alpha or interferon-alpha-Fc fusion protein.

Furthermore, it is contemplated that the specific effect of Fc-interferon-alpha is more pronounced in treatment of liver disease than in treatment of disorders localized to other tissues where Fc-interferon-alpha is not concentrated.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### Incorporation By Reference

The disclosure of each of the scientific articles and patent documents referenced to hereinabove is incorporated herein by reference.

1. A nucleic acid molecule encoding a fusion protein comprising:
(a) a signal sequence;
(b) an immunoglobulin Fc region; and
(c) a target protein sequence comprising interferon-alpha,
wherein the signal sequence, the immunoglobulin Fc region and the target protein sequence are encoded serially in a 5′ to 3′ direction.

2. The nucleic acid of claim 1 wherein the immunoglobulin Fc region comprises an immunoglobulin hinge region.

3. The nucleic acid of claim 1 wherein the immunoglobulin Fc region comprises an immunoglobulin hinge region and an immunoglobulin heavy chain constant region domain.

4. The nucleic acid of claim 1 wherein the immunoglobulin Fc region comprises an immunoglobulin hinge region and an immunoglobulin CH3 domain.

5. The nucleic acid of claim 1, wherein the immunoglobulin Fc region comprises a hinge region, a CH2 domain and a CH3 domain.

6. The nucleic acid of claim 5 wherein the immunoglobulin Fc region comprises a portion of an immunoglobulin gamma sequence.

7. The nucleic acid of claim 6 wherein the immunoglobulin gamma is human immunoglobulin gamma1.

8. A replicable expression vector for transfecting a mammalian cell, the vector comprising the nucleic acid of claim 1.

9. The replicable expression vector of claim 8 wherein the vector is a viral vector.

10. A mammalian cell harboring the nucleic acid of claim 1.

11. A fusion protein comprising in an amino terminal to carboxy terminal direction an immunoglobulin Fc region and a target protein comprising interferon-alpha.

12. The fusion protein of claim 11 wherein the interferon-alpha comprises an amino acid sequence set forth in SEQ. ID. NO.: 2, 7 or 8-21 or a species or allelic variant thereof.

13. The fusion protein of claim 11 wherein the target protein comprises at least two interferon-alpha molecules linked by a polypeptide linker.

14. The fusion protein of claim 13 further comprising a polypeptide linker linking the immunoglobulin Fc region to the target protein.

15. The fusion protein of claim 11 wherein the immunoglobulin Fc region comprises an immunoglobulin hinge region and an immunoglobulin heavy chain constant region domain.

16. The fusion protein of claim 15, wherein the heavy chain constant region domain comprises a CH3 domain.

17. The fusion protein of claim 11 wherein the immunoglobulin Fc region comprises a hinge region, a CH2 domain and a CH3 domain.

18. A multimeric protein comprising at least two fusion proteins of claim 11 linked via a covalent bond.

19. The protein of claim 18 wherein the covalent bond is a disulfide bond.

20. A method of producing a fusion protein comprising the steps of:
(a) providing the mammalian cell of claim 10; and
(b) culturing the mammalian cell to produce the fusion protein.

21. The method of claim 20 comprising the additional step of collecting the fusion protein.

22. The method of claim 20 comprising the additional step of purifying the fusion protein.

23. The method of claim 20 comprising the additional step of cleaving with a proteolytic enzyme the immunoglobulin Fc region from the target protein at a proteolytic cleavage site disposed between the immunoglobulin Fc region and the target protein.

24. A method of treating a condition alleviated by the administration of interferon-alpha comprising the step of administering the nucleic acid of claim 1 to a mammal having the condition.

25. A method of treating a condition alleviated by the administration of interferon-alpha comprising the step of administering the vector of claim 8 to a mammal having the condition.

26. A method of treating a condition alleviated by the administration of interferon-alpha comprising the step of administering the fusion protein of claim 11 to a mammal having the condition.

27. A method of treating a condition alleviated by the administration of interferon-alpha comprising the step of administering protein of claim 18 to a mammal having the condition.

28. The method of claim 26 wherein the condition is a liver disorder.

29. The method of claim 28 wherein the liver disorder is hepatitis.
